(19) United States
(12) Patent Application Publication
(10) Pub. No.: US 2004/0096903 A1
(43) Pub. Date: May 20, 2004

(54) Immobilised cardiolipin probes

(76) Inventors: William Kenneth Lang, Kent (GB); Andrew Bruce Holmes, Cambridge (GB); Ze-Yi Lim, Singapore (SG); Stuart John Conway, Cambridge (GB); Melloney Kate Johns, Cambridge (GB)

Correspondence Address: HELLER EHRMAN WHITE & MCAULIFFE LLP, 1666 K STREET,NW, SUITE 300, WASHINGTON, DC, 20006, US

(21) Appl. No.: 10/468,264

(22) PCT Filed: Feb. 20, 2002
(86) PCT No.: PCT/GB02/00720

(30) Foreign Application Priority Data
Feb. 20, 2001 (GB) 0104057.5

(51) Int. Cl. G01N 33/53; C08G 63/48; C08G 63/91

(52) U.S. Cl. 435/7.1; 525/54.3

(57) ABSTRACT

Probes comprising a cardiolipin derivative covalently attached to a solid phase, other than through an allylic oxygen, are described. Methods of making the probes are also described. A cardiolipin analogue which is amino-derivatised at the end of one of the fatty acid side chains is reacted with an activated ester attached to a solid support. The probes are useful for diagnosis of anti-phospholipid antibody syndrome (APS), and for identifying and purifying cardiolipin binding proteins.

This invention relates to diagnosis of antiphospholipid antibody syndrome (APS), to probes for use in the diagnosis, and to methods of making the probes. The probes are also useful for identifying and purifying proteins which bind selectively to cardiolipin.

Antiphospholipid antibodies, including anticardiolipin antibodies, are frequently detected in sera from patients with systemic lupus erythematosus (SLE) and other related autoimmune disorders. These autoantibodies have been associated with various venous and arterial thrombotic disorders, including cerebral or myocardial infarction, deep venous thrombosis, thrombocytopenia, pulmonary embolism and recurrent foetal lose due to placental infarction. The term antiphospholipid antibody syndrome (APS) has been applied to such disorders. Lupus anticoagulant has also been associated with APS, although it is not thought to be identical to anticardiolipin antibody.

In order to assess the risk of APS in individuals with SLE or related disorders, it is known to test the serum of such individuals for the presence of antibodies to cardiolipin by enzyme immunoassay, for example using the RELISA CARDIOLIPIN in vitro diagnostic test of Immuno Concepts. In this test a patient serum sample is diluted in buffer containing apolipoprotein H cofactor and added to a microwell coated with cardiolipin. The cofactor is thought to be required for binding of anticardiolipin antibody to cardiolipin. Anti-cardiolipin antibodies in the sample which bind to the cardiolipin are then detected using anti-human antibody labelled with horseradish peroxidase and a solution of tetramethylbenzidine (TMB) and hydrogen peroxide as a chromogenic substrate.

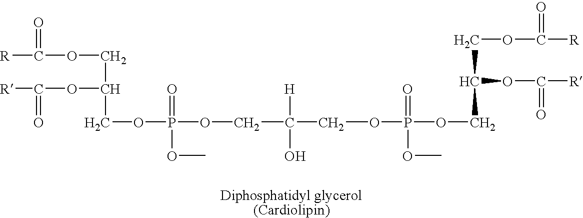

In such types of assays it is desirable to use a detergent to reduce non specific binding and hence increase assay sensitivity and specificity. However, detergent can remove non covalently immobilised cardiolipin from the solid phase. It is preferred, therefore, to use probes comprising a solid phase to which cardiolipin is covalently attached. This is also advantageous for identifying and purifying proteins which bind to cardiolipin because once the bound proteins have been removed, the probe can be re-used.

WO 91/10138 (Baxter Diagnostics) refers to methods of covalently immobilising cardiolipin to a solid phase and use of the immobilised cardiolipin to detect the presence of anti-cardiolipin antibodies. On page 4, line 31 to page 5, line 5, methods of covalently immobilising cardiolipin via the polar head group and/or fatty acid moieties are listed as:

i) SeO₂ oxidation

ii) PCC Oxidation

iii) m-chloroperbenzoic acid oxidation

iv) 1,4-butanediol diglycidyl ether (oxirane coupling)

v) Biotin coupling by EDC

vi) Succinic anhydride coupling.

The function of all these reagents in the coupling procedure can be determined, for example, by consulting the series “Fieser and Fieser's Reagents for Organic Synthesis”, Volumes 1-12, 1967-1986, Ed. Mary Fieser, Wiley, N.Y. Selenium dioxide effects oxidation at allylic positions, thereby converting an alkene to an allylic alcohol (OH introduced in place of H at a carbon adjacent to an alkene carbon). PCC (pyridinium chlorochromate) oxidises primary and secondary alcohols to the corresponding carbonyl compounds. m-Chloroperbenzoic acid (MCPBA) converts alkenes to epoxides (oxiranes) which can undergo nucleophilic ring opening. 1,4-butane diol diglycidyl ether contains terminal epoxides which could undergo nucleophilic ring opening reactions to provide a linker between a substrate and a solid phase. Biotin is a bicyclic heterocyclic molecule terminating in a five carbon chain carboxylic acid. EDC is used to link amino groups via an amide bond to biotin which has strong non-covalent affinity to other natural molecules is such as avidin and streptavidin. Succinic anhydride can be ring-opened to form amide or ester links terminating in a carboxylic acid which can be coupled to another amide or ester. It is therefore a linker molecule.

It should be noted that natural cardiolipin is a diphosphatidyl glycerol in which the fatty acid side chains (R, R′ in the cardiolipin structure shown above) may be selected from a wide variety of naturally occurring fatty acids. Examples of this selection are:

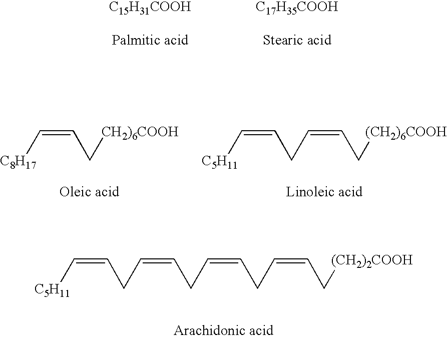

### Selection of Fatty Acids for Cardiolipin

Only selenium dioxide, pyridinium chlorochromate, and m-chloroperbenzoic acid are relevant to the question of functionalising the fatty acid residues in natural cardiolipin. Covalent linkage of cardiolipin using these reagents is summarised below:

1. Covalent Linkage Using Selenium Dioxide and Subsequent Oxidation with PCC

Cardiolipin is treated with selenium dioxide (SeO₂) which effects oxidation at any allylic position to yield an allylic alcohol. Any position which is allylic (i.e. a- to a carbon-carbon double bond) is oxidised.

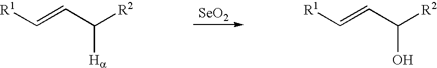

### Selenium Dioxide Effected Allylic Oxidation

Pyridinium chlorochromate could oxidise such an allylic alcohol to the corresponding α,β-unsaturated ketone, possibly with an allylic transposition. This reagent can also convert alkenes directly into the α,β-unsaturated ketone, again with allylic transposition.

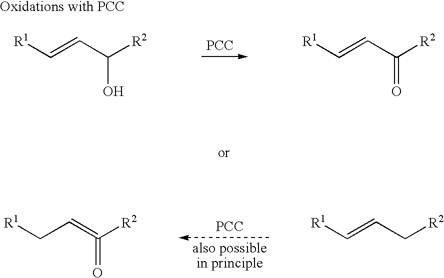

No description is given or implied in WO 91/10138 as to what is the PCC oxidation product is or how the unsaturated ketone might be coupled to a support. One might conceive a conjugate addition of a nucleophile to the β-carbon of the α,β-unsaturated ketone. If the starting fatty acid had an OH in the side chain PCC would oxidise this to a ketone, but no method of coupling the ketone is described.

2. Covalent Linkage Using m-Chloroperbenzoic Acid (MCPBA)

MCPBA can form an epoxide (oxirane) at a C═C. The resulting oxirane could be ring-opened by nucleophilic attack.

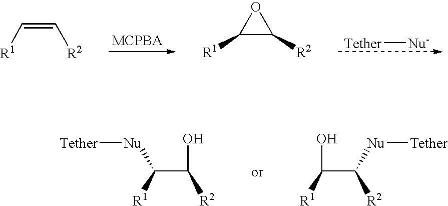

### Epoxidation of Alkene and Opening with a Tethered Nucleophile

All the above reactions allow covalent linkage of cardiolipin to be effected through the fatty acid moieties. The derivatised cardiolipin is reacted (via the allylic alcohol) with a carbamoyl moiety linked to a solid support in a 1-ethyl-3 (3-dimethyloaminopropyl) carbodiimide (EDC) mediated coupling reaction.

There are several disadvantages of use of selenium dioxide and subsequent oxidation with PCC to attach cardiolipin to a solid support:

1. It is only possible to form a link at an allylic position. Thus, the cardiolipin must have an unsaturated linkage (for allylic oxidation) or an adventitious alcohol substituent in the chain (for PCC oxidation), but it is not obvious how this would enable coupling to a bead.

2. Any allylic position will be oxidised so there is no control over the location of the point of attachment unless the cardiolipin used only has a single allylic position.

3. Natural cardiolipin is a mixture of different cardiolipins having different fatty acid moieties and so is likely to contain many double bonds. In this case, all the allylic positions will be oxidised, so links can form between any allylic alcohol and the solid support. Multiple different cardiolipin species will then be attached to the solid support, with each different cardiolipin species being attached at a different part of the cardiolipin molecule. Probes with cardiolipin immobilised in this way are unlikely to be useful in diagnosis and it will be difficult to use these probes to purify and identify proteins that bind specifically to cardiolipin.

Similar disadvantages arise with m-chloroperbenzoic acid oxidation: the cardiolipin must have an alkene group (in the fatty acid) in order to be epoxidised; epoxidation is unspecific if more than one alkene is present in the fatty acid; and ring opening of any epoxide is unspecific if this is used to tether the molecule to a solid phase.

Thus, if the methods of coupling disclosed in WO 91/10138 are to be used to covalently immobilise cardiolipin for use in diagnosis of APS or to affinity purify proteins which bind specifically to cardiolipin, it is necessary to ensure that only one species of cardiolipin is used with only one double bond. It is desired to provide improved methods for covalent immobilisation of cardiolipin and improved cardiolipin probes.

According to the invention there is provided a probe comprising a cardiolipin derivative covalently attached to a solid phase other than through an allylic oxygen.

It is preferred that the cardiolipin derivative is not coupled to the solid phase by a linker arising from functionalising an α,β-unsaturated ketone by conjugation addition, or by ring-opening of an epoxide:

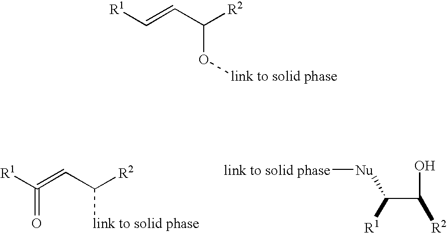

Preferably, the probe has the following general formula:

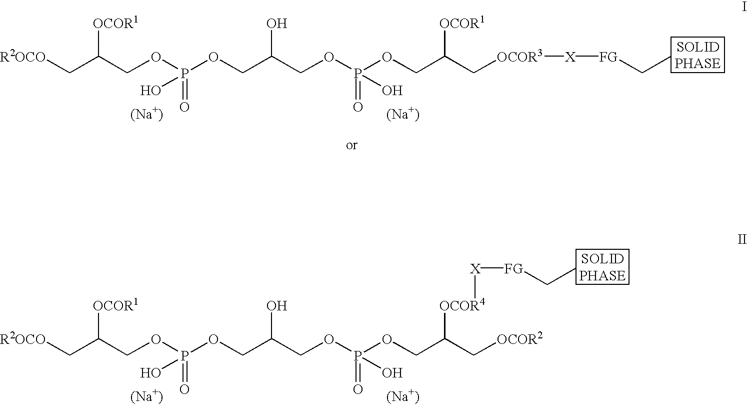

R1, R2, R3, R4 are alkyl, preferably C₅-C₁₆ alkyl.

Unsaturations are allowed;

X is O, S, or preferably NH

FG comprises carbonyl from a carboxylate (thiolo)ester, or preferably an amide.

Any suitable covalent attachment may link the solid phase to the functional group. It is to be noted that this symbolic illustration is not to be interpreted as representing solely a —CH₂— linkage between the functional group and the solid phase.

A preferred probe has the following formula:

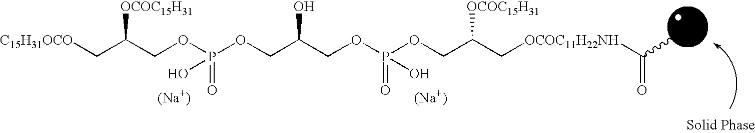

The symbolic illustration showing the link between the —C═O and the solid phase does not necessarily represent the chemical structure of this link. Any suitable covalent attachment may be used.

The solid phase may be any suitable solid phase on which binding reactions to the cardiolipin derivative of the probe may be carried out. Preferred examples are ELISA plates and beads, such as agarose or sepharose beads. Beads are particularly advantageous because they can be readily manipulated thereby allowing binding, washing, and detection reactions to be easily carried out.

There is also provided a method of making a probe of the invention in which a cardiolipin analogue of formula I′ or II′:

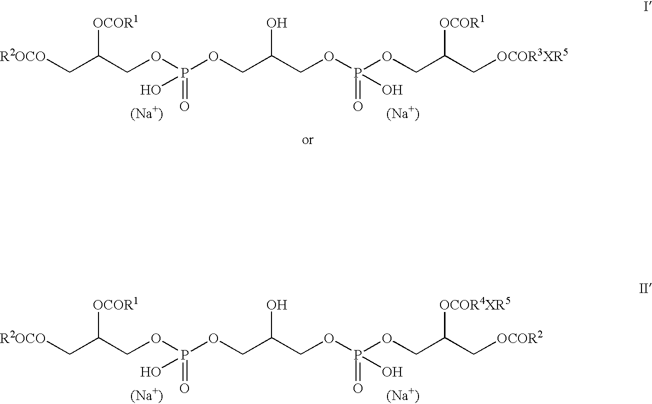

is reacted with: RG-SOLID PHASE

Where:

R1, R2, R3, R4 are alkyl, preferably C₅-C₁₆ alkyl.

Unsaturations are allowed.

X is NH, O, or S

RG is a reactive group, coupled to the solid phase, which is capable of reaction with the —XH group of the cardiolipin analogue to thereby covalently couple the analogue to the solid phase. RG is preferably an activated ester, e.g. N-hydroxysuccinimide (NHS)-activated carboxylate. RG may be coupled to the solid phase by any suitable covalent attachment.

R5 is H or a protecting group.

A preferred method comprises carrying out one of the following reactions:

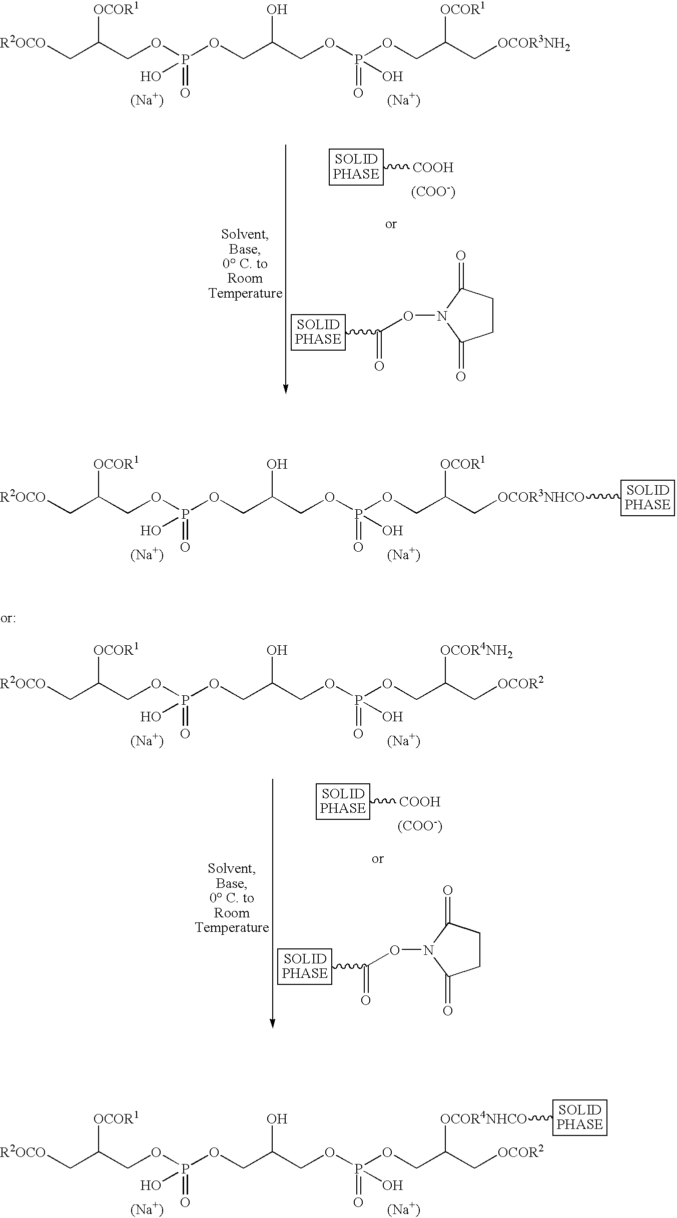

R1, R2, R3, R4 are alkyl, preferably C₅-C₁₆ alkyl. Unsaturations are allowed. The solvent is preferably anhydrous alcohol, DMSO, or water. The base is preferably NaHCO₃. The preferred temperature is about 0° C., except when the solvent is DMSO in which case the preferred temperature is about 20° C.

Preferred methods of making a probe of the invention comprise the steps shown in reaction scheme 3 or 4.

Probes and methods of the invention have many advantages:

There is no requirement for any of the fatty acid groups of the cardiolipin derivative or analogue to include a carbon-carbon double bond, and there is no non-specific coupling to the solid phase even if more than one carbon-carbon double bond is present in the fatty acid groups. Only one species of cardiolipin derivative is attached to the solid support at a known position. Probes of the invention are thus ideal for identifying proteins which bind selectively to the attached cardiolipin derivative, and for use as diagnostic tools.

It is possible to select any chain length between the head group of the cardiolipin derivative and the solid support. The required length of the lipid chain is selected before synthesis. This is important because certain chain lengths may be required to sufficiently space the polar head group of the cardiolipin derivative from the solid support in order to best mimic natural cardiolipin. Different chain lengths can be tested to identify the optimum length for binding of particular proteins. This will allow optimum binding of proteins which bind specifically to natural cardiolipin and thus improve diagnosis and protein isolation using the probes.

It is possible to estimate the percentage loading of the cardiolipin derivative on the solid phase.

If desired, a probe of the invention may comprise a cardiolipin derivative in which the carbon chains of the fatty acid moieties are all saturated. This is in contrast to the immobilised cardiolipin produced according to the methods disclosed in WO 91/10138 in which at least one carbon-carbon double bond must be provided.

The conditions for coupling a cardiolipin analogue to a solid phase according to the invention are milder than the conditions for covalent coupling disclosed in WO 91/10138.

Also provided according to the invention are cardiolipin analogues having the following general formula:

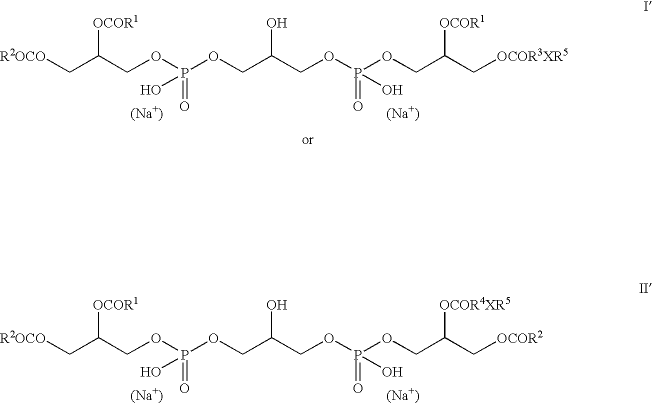

R1, R2, R3, R4 are alkyl, preferably C₅-C₁₆ alkyl.

Unsaturations are allowed.

R5 is H or a protecting group,

X is NH, O, or S

There is also provided according to the invention use of a cardiolipin analogue in the production of a probe of the invention.

Preferred methods of making a cardiolipin analogue of the invention comprise the steps shown in reaction scheme 3 or 4.

There is also provided use of a probe of the invention for diagnosing susceptibility to a disease or disorder, or for diagnosis of a disease or disorder, such as APS. Where the probe is used for the diagnosis of APS, the cardiolipin derivative may be any derivative of cardiolipin which can be bound by anticardiolipin antibody in the presence of any cofactor required for binding of cardiolipin by anticardiolipin antibody.

A cofactor thought to be required for binding of anticardiolipin antibody to cardiolipin is β₂-glycoprotein I (apolipoprotein H) [Koike and Matsuura, E.L.E.F. CARING AND SHARING, Newsletter 4].

There is further provided according to the invention a method of assaying for the presence of anti-cardiolipin antibody in a sample, the method comprising contacting the sample with a probe of the invention under conditions which permit binding of anti-cardiolipin antibody to the probe, and detecting for the presence of anti-cardiolipin antibody bound to the probe.

Typically, the sample to be tested will be a patient serum sample (possibly diluted). Although any cofactor required for binding of anti-cardiolipin antibody to cardiolipin may be present in the sample, it may be preferable to add a cofactor (such as apoliprotein H) to the sample in order to ensure that sufficient cofactor is present to allow binding of any anticardiolipin antibody in the sample to the cardiolipin derivative of the probe.

It is possible that other antiphospholipid antibodies may be capable of binding to the cardiolipin derivative of the probe. Binding of these antibodies may also be cofactor dependent. A paper from the Pathology Bulletin Board (Velan, Re: Lupus Anticoagulant) states that antiphosphilipid antibodies bind to proteins bound to anionic phosphlipids (e.g. beta 2-glycoprotein I, prothrombin, protein C). Consequently, it may be preferable to add such cofactors to the sample to assay for the presence of other antiphospholipid antibodies in the sample.

Detergent may be used in assays of the invention to reduce non specific binding to the probe. Where the probe comprises a cardiolipin derivative covalently attached to beads or other microparticles, detergent may be used to enhance the solubility of the beads/microparticles.

The invention also provides use of a method of assaying for anti-cardiolipin antibody and/or other antiphospholipid antibody in a sample for assessing the susceptibility of an individual to APS, or for diagnosing an individual with APS.

The invention also provides a kit for assaying for the presence of anticardiolipin and/or other antiphospholipid antibody in a sample which comprises a probe of the invention, and a means for detecting anticardiolipin antibody and/or other antiphospholipid antibody bound to the probe.

Preferably the detection means comprises an anti-human antibody coupled to an enzyme and a chromogenic or fluorogenic substrate for the enzyme. A preferred enzyme is horseradish peroxidase and a preferred chromogenic substrate is TMB. Other suitable detection means include radiolabelled anti-human antibody.

A kit of the invention may further include suitable buffers required for carrying out assays using the probe and detection means of the kit.

Probes of the invention can also be used to identify and/or isolate proteins which bind to cardiolipin. In order to efficiently identify such proteins, it is advantageous if the probes can bind proteins which are present in relatively low abundance and/or proteins which have relatively low cardiolipin affinity. The physical characteristics of the covalent linkage of the cardiolipin derivative to the solid phase are thought to be an important factor in binding of relatively low abundance and/or low affinity proteins.

In particular, it is thought that attachment of the cardiolipin derivative via a long-chain fatty acid side chain of the molecule to the solid phase ensures that the head group of the cardiolipin derivative is available for binding by a cardiolipin binding protein. It is believed that this arrangement mimics cellular cardiolipin. It is thought that the length of the linkage between the head group and the solid phase should not be too short, otherwise the solid phase may sterically interfere with binding. A suitable length for the alkyl part of the fatty acid side chain is about C₅₋₁₆.

The invention provides an assay method which involves the step of detecting and/or measuring the binding of a probe of the invention when said probe is exposed to a protein in a test sample. Such an assay may involve the steps of identifying and/or isolating said protein by binding to said probe. Said probe may be used to detect/measure/identify and/or isolate more than one type of cardiolipin binding protein from a test sample containing many proteins. More than one type of probe may be used to detect/measure/identify and/or isolate more than one type of cardiolipin binding protein. The test sample may be a tissue or tissue culture extract, preferably a lysed extract. The test sample may be obtained by lysis of cells in a buffer containing at least one non-ionic surfactant, such as TRITON (RTM) X-100 or NP-40. The probe may be exposed to said test sample in the presence or absence of soluble cardiolipin. Protein-probe binding may be compared between more than one test sample to determine cardiolipin binding protein variation between said samples.

There is also provided: use of an assay method of the invention to detect/measure/identify and/or isolate a cardiolipin binding protein in a test sample; use of an assay method of the invention to detect and/or measure the ability of an agent, applied to said cardiolipin binding protein-containing test sample, to agonise or antagonise protein-probe binding; use of an assay method of the invention to detect and/or measure the ability of an agent, applied to said probe, to agonise or antagonise protein-probe binding.

The invention further provides a cardiolipin binding protein detected/measured/identified and/or isolated by an assay method of the invention, and an agent capable of agonising or antagonising protein-probe binding detected and/or measured by use of an assay method of the invention.

In a further embodiment, a probe of the invention may be modified to carry a photoaffinity label such as aryl azides, α-halo-carbonyl compounds, diaryl ketones. Such probes can be used to map the binding pocket of a cardiolipin binding protein. A fluorescent reporter group could be attached to a probe to obtain binding affinities.

In a further aspect of the invention, a probe of the invention coupled to scintillant may be used to identify an agonist or antagonist of the interaction of a cardiolipin binding protein with cardiolipin. Such uses are particularly suited for high throughput screening of candidate agonists/antagonists, especially single step high throughput screening. A radiolabelled protein (radiolabelled for example with tritiated leucine, or ³⁵S-methionine) known to bind cardiolipin is tested for binding to a probe of the invention coupled to scintillant in the presence and absence of one or more candidate agonists and/or antagonists. The advantage of using probe coupled to scintillant is that the difference in signal obtained between normal binding (i.e. in a control sample without any candidate antagonist or agonist) of cardiolipin binding protein to the probe and reduced or enhanced binding (i.e. in samples with agonist or antagonist) is much greater than can be obtained without the scintillant. Consequently, agonists and antagonists can be more readily identified. A similar strategy but using fluorescence detection can be envisaged, with the probe and the protein containing fluorophores of different excitation.

A general approach for identifying cardiolipin binding proteins from tissue extracts is as follows: The tissue is homogenised using standard methods, and two fractions are produced, cytosol and membranes. The cytosol fraction is mixed 1:1 with buffer A (50 mM Tris-HCl pH 8.0, 150 mM NaCl, 10 mM EDTA, 1% NP-40, protease inhibitors) and then incubated with a probe of the invention equilibrated for 30 min in buffer B (50 mM Tris-HCl pH 7.5, 150 mM NaCl, 5 mM EDTA, 0.1% Tween-20, 0.02% Na azide). The membrane fraction is mixed 1:3 with buffer A but containing 2% NP-40 for 30 is min on ice. The sample is then spun at 100,000 Xg for one hour to produce a soluble membrane extract. This extract is mixed with cardiolipin beads (i.e. probes of the invention in which the solid phase is a bead) equilibrated as described above and processed similarly as above. The sample is put in a rotator at 4 C for 2 hr, and then washed three times with buffer B in the cold. These washes are very important since they remove non-specifically bound proteins. To provide an extra level of specificity we do the following modification. To one of duplicate samples excess soluble cardiolipin is added before the beads are introduced (the soluble cardiolipin solution is made by drying C:12 or C:8 cardiolipin dissolved in chloroform, resuspending in buffer A and sonicating for 5 min to make a stock solution of 250 mM). The assumption is that excess soluble cardiolipin will compete with the cardiolipin on the beads thus reducing the amount of protein that is recovered bound to the beads (see FIG. 1). Bands of interest are excised from the gel and treated with trypsin. The tryptic digests produced from the various bands are analysed by mass spectroscopy.

Cardiolipin binding proteins obtainable using the invention are expected to fall into three categories: proteins of known identity and function but whose exact mechanism of action is not well understood, proteins of known identity but whose function is not understood, and totally novel proteins.

The probes of the invention are general analytical tools for identification of cardiolipin binding proteins from different tissues and biological fluids. We envisage that the cytosolic and membrane contents of any cell type can be screened for cardiolipin binding proteins using these probes (in addition to brain, a partial list includes liver, kidney, heart, pancreas, macrophages, neutrophils). In all cases, cytosolic or membrane fractions could be subjected to assays as described above. Once a series of proteins, which bind directly to cardiolipin have been identified, they could be examined as to which amino acids are involved in the binding, using a photoaffinity labeled cardiolipin analogue. Comparison among those proteins should result in a common motif which may define a cardiolipin binding motif. Once the motif is identified, it can be used as a search tool to identify most proteins, that are expected to bind cardiolipin and that are described in the databases. Thus the probes are expected to reveal the majority of the members of the cardiolipin binding protein families.

We foresee important applications of the probes in diagnostics. Extracts from healthy or pathological tissues could be compared side by side and their full complement of cardiolipin binding proteins may hence be established. Any protein whose amount and/or electrophoretic mobility changes in the pathological tissue in comparison to the healthy tissue could be identified by mass spectroscopy. Such proteins will be candidates both as markers for the disease and as therapeutic targets (see below).

The approach of identifying candidate proteins by comparing their expression level and pattern between “normal” and “altered” tissues or cell lines has similarity to current proteomics strategies that are in use by many pharmaceutical companies whereby total cellular proteins from such tissues are analysed with a view to identify potentially interesting changes in expression profiles. We point out two essential differences with the approach proposed here: (a) The probe of the invention acts as a concentration/enrichment reagent thus allowing small differences, or differences in rare proteins to be more readily detectable. (b) Since a functional requirement is built into the screening process (i.e. cardiolipin binding), the resulting proteins from our approach can be studied with some prior knowledge of their potential function.

We foresee important applications of the probes of the invention in therapeutics. The probes provide unique tools for identification of small molecule compounds that interfere with or enhance cardiolipin binding of proteins since they are amenable to automated assays. Following identification of a candidate target protein, specific monoclonal antibodies against this protein could be raised and the protein itself may then be produced in miligram amounts. The preferred binding assay is based on detection by ELISA using the specific antibodies raised. Other configurations of the binding assay include the use of cardiolipin functionalised with a fluorescent reporter group (detection of binding will be done by fluorometry) or the use of radioactive protein (detection of binding will be done by scintillation counting). Candidate compounds (obtained from commercial sources) can be introduced in the binding assay prior to adding the probe. If a compound interferes with binding, detection of the protein is expected to be reduced. If it enhances binding, detection should be higher. Compounds identified using this screen might become interesting drug lead candidates.

Whilst the length of the fatty acid side chain that links the cardiolipin derivative to the solid phase may be chosen to mimic the natural presentation of the head group of cardiolipin to proteins in the cell, the length of the chain may instead be chosen deliberately to result in a non-natural presentation.

There is also provided according to the invention use of a probe comprising a cardiolipin derivative covalently attached to a solid phase to identify and/or isolate a cardiolipin binding protein. Preferably the binding protein is not an antibody.

Further embodiments of the invention are now described, by way of example only.

Cardiolipin beads of the following formula may be synthesised according to the reaction schemes shown in example 1 or 2:

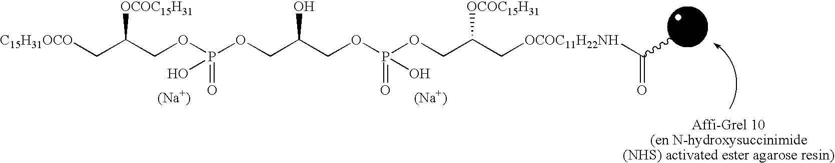

### EXAMPLE 1

Synthesis of Cardiolipin Beads (Method 1)

Synthesis of Fragment 5:

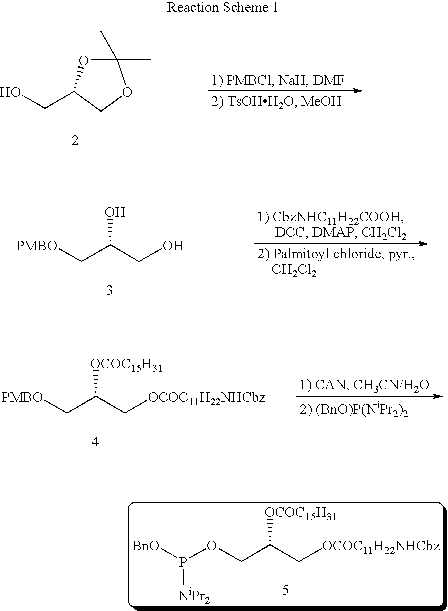

Synthesis of Fragment 8:

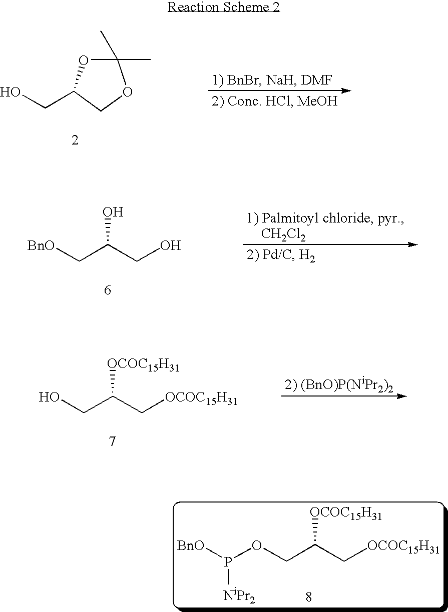

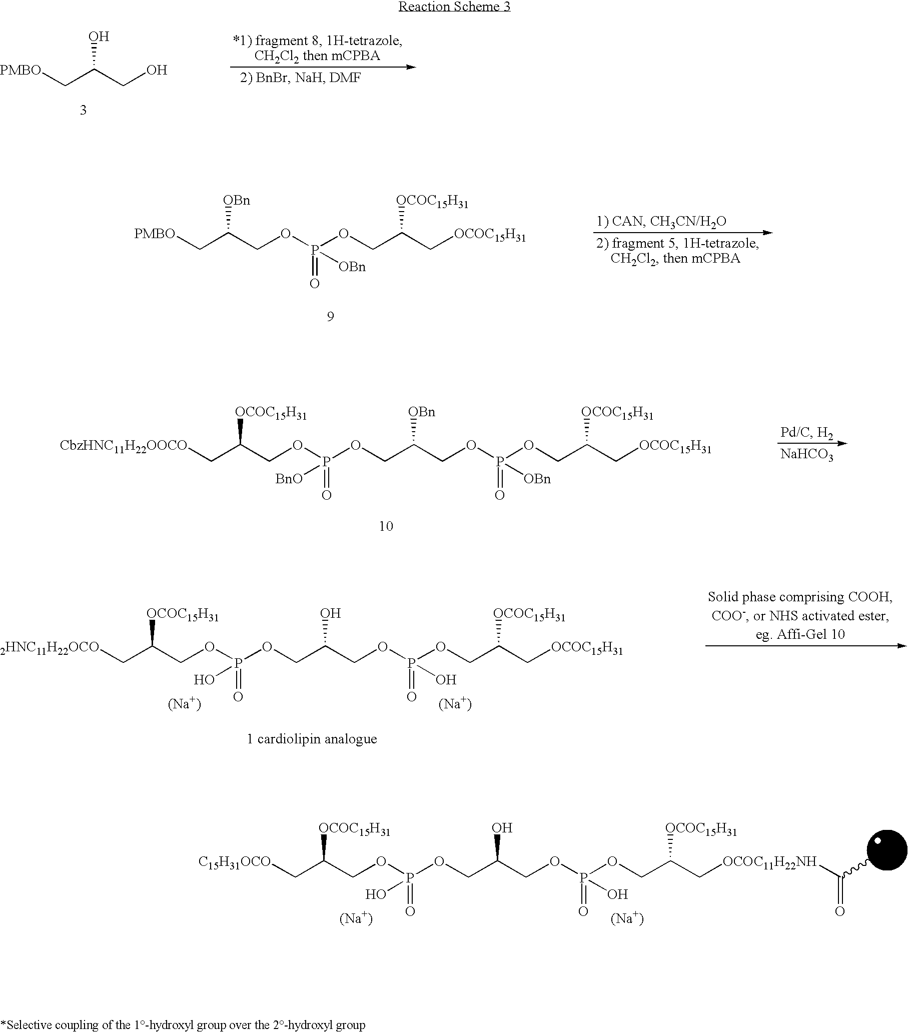

### EXAMPLE 2

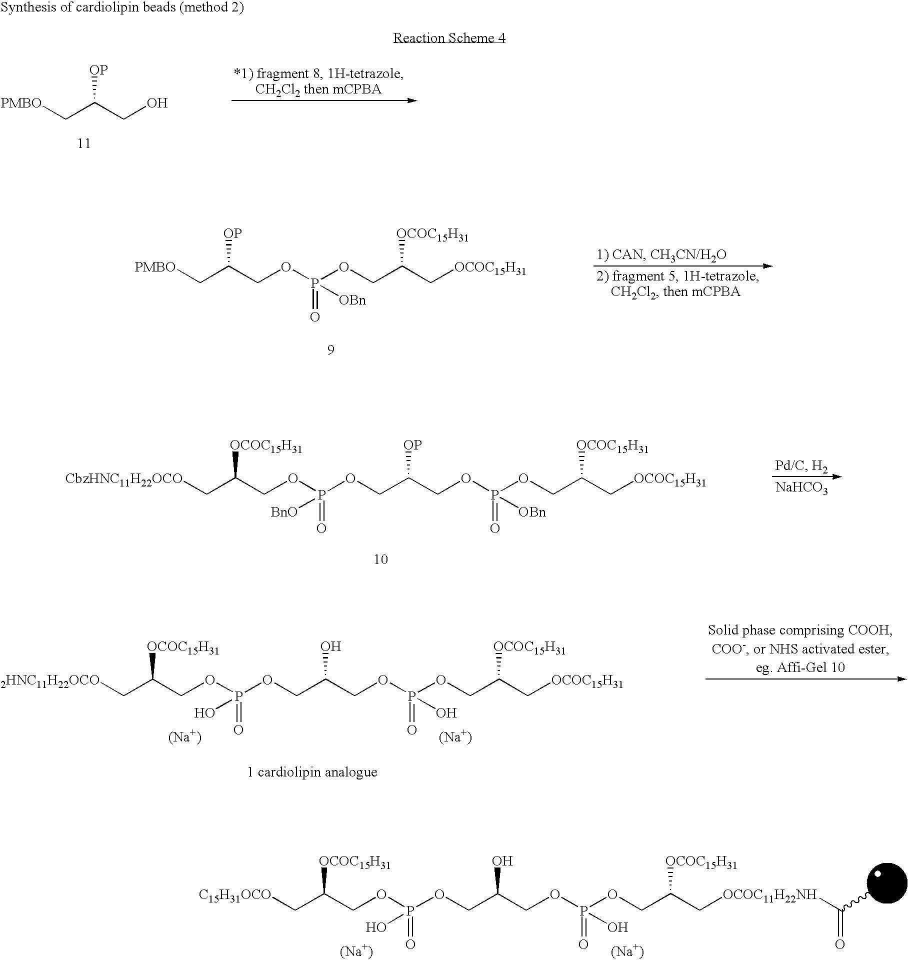

### EXAMPLE 3

Assaying for Anticardiolipin Antibody

A suitable method for assaying for the presence of cardiolipin antibody in a serum sample is outlined below. Such a method may be used to diagnose an individual with APS or with susceptibility to APS.

1. A serum sample suspected of containing anticardiolipin antibodies is added to cardiolipin beads (made as described in example 1 or 2) in a buffer of suitable concentration and pH to permit binding of anticardiolipin antibody to the cardiolipin derivative of the beads. The buffer contains apolipoprotein H cofactor in order to ensure that there is sufficient cofactor present to allow optimal binding of anticardiolipin antibody to the beads.

2. The serum sample, beads, and buffer are incubated for 30-60 minutes at about 30° C. to allow binding of anticardiolipin antibody to the cardiolipin derivative of the beads.

3. The beads are then washed in wash buffer to remove unbound antibody and other serum proteins from the beads.

4. The washed beads are incubated with anti-human antibody labelled with horseradish peroxidase under standard conditions (these are well known to a person of ordinary skill in the art).

5. Anticardiolipin antibody bound to the cardiolipin beads can then be detected using tetramethylbenzidine (TMB) and sulphuric acid as chromogenic substrate (again, under standard conditions which are well known to those of ordinary skill in the art).

1. A probe comprising a cardiolipin derivative covalently attached to a solid phase other than through an allylic oxygen.

2. A probe according to claim 1 having the following general formula: 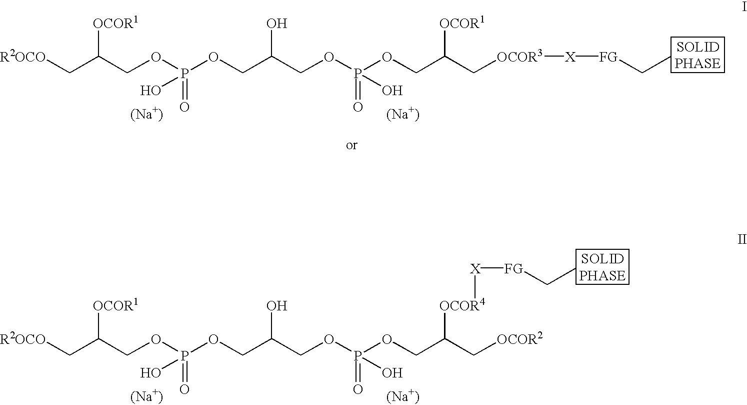
R1, R2, R3, R4 are alkyl, preferably C₅-C₁₆ alkyl.
Unsaturations are allowed;
X is O, s, or preferably NH;
FG comprises carbonyl from a carboxylate (thiolo)ester, or preferably an amide.

3. A probe according to claim 2 having the following formula:

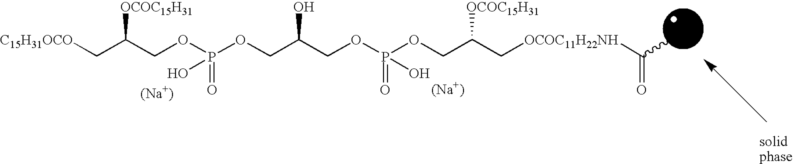

4. A probe according to any preceding claim wherein the is solid phase comprises beads, preferably agarose or sepharose beads.

5. A method of making a probe according to claim 1 which comprises reacting a cardiolipin analogue of formula I′ or II′: 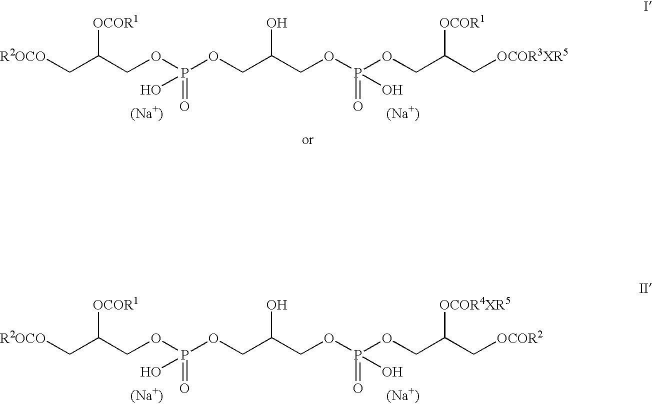
with:
RG-Solid Phase
Where:
R1, R2, R3, R4 are alkyl, preferably C₅-C₁₆ alkyl.
Unsaturations are allowed.
X is NH, O, or S.
R5 is H or a protecting group,
RG is a reactive group, coupled to the solid phase, which is capable of reaction with the —XH group of the cardiolipin analogue to thereby covalently couple the analogue to the solid phase. RG is preferably an activated ester, e.g. N-hydroxysuccinimide (NHS)-activated carboxylate.

6. A method of making a probe according to claim 2 which comprises carrying out the following reaction: 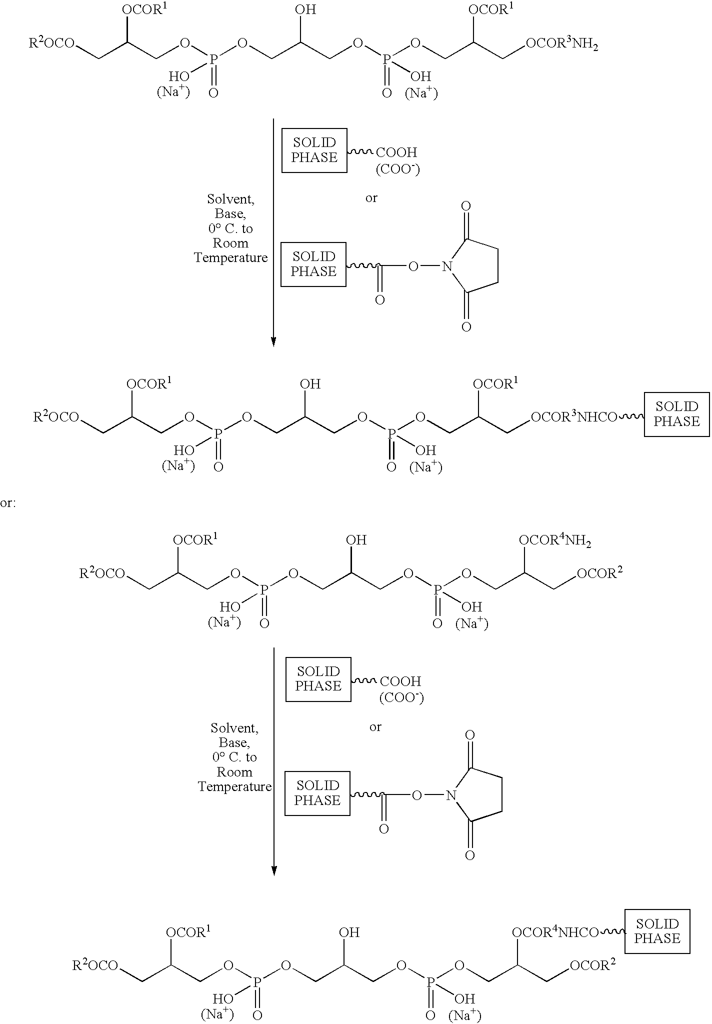
R1, R2, R3, R4 are alkyl, preferably C₅-C₁₆ alkyl.
Unsaturations are allowed.

7. A method of making a probe according to any of claims 1 to 4 which comprises the steps shown in reaction scheme 3 or 4.

8. A cardiolipin analogue having the following general formula: 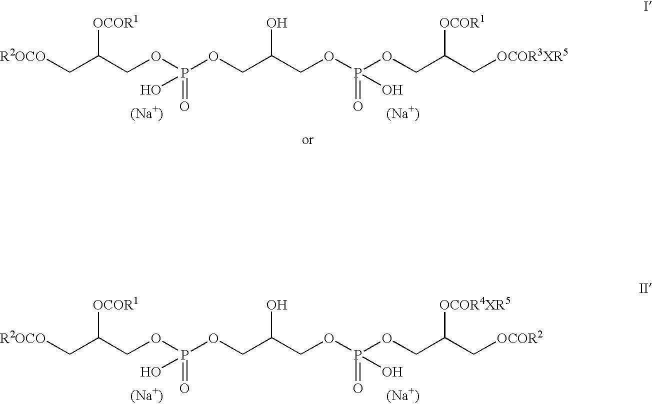
R1, R2, R3, R4 are alkyl, preferably C₅-C₁₆ alkyl.
Unsaturations are allowed.
R5 is H or a protecting group.
X— is NH, O, or S.

9. A cardiolipin analogue according to claim 8 having the following formula:

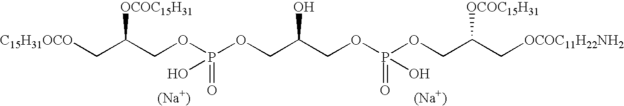

10. Use of a cardiolipin analogue according to claim 8 or 9 for the production of a probe according to any of claims 1 to 4.

11. A method of making a cardiolipin analogue according to claim 8 or 9 which comprises the steps shown in reaction scheme 3 or 4.

12. A method of assaying for the presence of anti-cardiolipin antibody and/or other antiphospholipid antibody in a sample, the method comprising contacting the sample with a probe according to any of claims 1 to 4 under conditions which permit binding of anti-cardiolipin antibody and/or other antiphospholipid antibody to the probe, and detecting for the presence of anti-cardiolipin antibody and/or other antiphospholipid antibody bound to the probe.

13. A method according to claim 12 in which cofactor required for binding of anti-cardiolipin antibody and/or other antiphospholipid antibody to cardiolipin is added to the sample.

14. Use of a method according to claim 12 or 13 for assessing the susceptibility of an individual to antiphospholipid antibody syndrome, or for diagnosing an individual with antiphospholipid antibody syndrome.

15. A kit for assaying for the presence of anticardiolipin antibody in a sample which comprises a probe according to any of claims 1 to 4, and a means for detecting anticardiolipin antibody bound to the probe.

16. A kit according to claim 15 in which the detection means comprises an anti-human antibody coupled to an enzyme and a chromogenic or fluorogenic substrate for the enzyme.

17. Use of a probe according to any of claims 1 to 4 to bind a binding partner of the cardiolipin derivative.

18. Use of a probe according to any of claims 1 to 4 to affinity purify a binding partner of the cardiolipin derivative.

19. Use of a probe according to any of claims 1 to 4 to test the cardiolipin binding activity and/or affinity of a protein.

20. An assay method which involves the step of detecting and/or measuring the binding of a probe according to any of claims 1 to 4 when said probe is exposed to a protein in a test sample.

21. An assay according to claim 20 which involves the step of identifying and/or isolating said protein by binding to said probe.

22. An assay according to claim 20 or 21 wherein said probe is used to detect/measure/identify and/or isolate more than one type of cardiolipin binding protein from a test sample containing many proteins.

23. An assay according to any of claims 20 to 22 wherein said test sample is a tissue or tissue culture extract, preferably a lysed extract.

24. An assay according to claim 22 wherein said test sample is obtained by lysis of cells in a buffer containing at least one non-ionic surfactant, such as TRITON (RTM) X-100 or NP-40.

25. An assay according to any of claims 20 to 24 wherein said probe is exposed to said test sample in the presence or absence of soluble cardiolipin.

26. An assay according to any of claims 20 to 25 wherein protein-probe binding is compared between more than one test sample to determine cardiolipin binding protein variation between said samples.

27. Use of an assay according to any of claims 20 to 26 to detect/measure/identify and/or isolate a cardiolipin binding protein in a test sample.

28. Use of an assay according to any of claims 20 to 26 to detect and/or measure the ability of an agent, applied to said cardiolipin binding protein-containing test sample, to agonise or antagonise protein-probe binding.

29. Use of an assay according to any of claims 20 to 26 to detect and/or measure the ability of an agent, applied to said probe, to agonise or antagonise protein-probe binding.

30. A cardiolipin binding protein detected/measured/identified and/or isolated by an assay according to any of claims 20 to 26.

31. An agent capable of agonising or antagonising protein-probe binding detected and/or measured by use of an assay according to claim 28 or 29.
